(19) United States
(12) Patent Application Publication
(10) Pub. No.: US 2023/0386227 A1
(43) Pub. Date: Nov. 30, 2023

(54) DEEP LEARNING ANOMALY DETECTION SYSTEMS FOR RESPONDING TO EMERGENCY EVENTS IN VEHICLES

(71) Applicant: MEILI TECHNOLOGIES, INC., New York, NY (US)

(72) Inventors: Samantha Nicole LEE, Bronx, NY (US); John Joseph DEFELICE, Nashua, NH (US); Christopher KANAN, New York, NY (US); Wendy JU, New York, NY (US)

(73) Assignee: MEILI TECHNOLOGIES, INC., New York, NY (US)

(21) Appl. No.: 18/323,253

(22) Filed: May 24, 2023

Related U.S. Application Data
(60) Provisional application No. 63/345,428, filed on May 25, 2022.

(51) Int. Cl. G06V 20/59; G06V 10/77; G06V 40/10; B60W 60/00; B60W 50/14; A61B 5/00

(52) U.S. Cl. CPC G06V 20/59; G06V 10/7715; G06V 40/10; B60W 60/001; B60W 50/14; A61B 5/6893; B60W 2420/42; B60W 2540/221; B60W 2556/10

(57) ABSTRACT

Vehicle occupant anomaly detection is provided. A system can receive sensor data from sensors associated with a vehicle, the sensors including an imaging sensor and the sensor data including 3D point representations of a vehicle occupant. The system can extract a time-series features of the vehicle occupant from the 3D point representations. The system can execute a machine learning model using the time-series features to determine at least one condition of the vehicle occupant. The system can, responsive to the condition, generate an instruction to cause the vehicle to perform a navigational action.

### CROSS-REFERENCE TO RELATED APPLICATIONS

This application claims priority to U.S. Provisional Patent Application Ser. No. 63/345,428, filed May 25, 2022, which is incorporated by reference in its entirety.

### TECHNICAL FIELD

Embodiments of the invention are in the field of deep learning-based anomaly detection and pertain particularly to systems and method for detecting emergency events in a vehicle and responding to these emergency events to provide emergency assistance.

### BACKGROUND

The smart vehicle-based sector is recognizing the benefits of deep learning software solutions and has started to incorporate those technologies into smart vehicles and related infrastructure. Deep learning, coupled with hardware such as sensors, has already advanced the capabilities of autonomous and semiautonomous vehicles, where, for example, models trained through deep learning algorithms automate or simplify the decision-making processes regarding the operation and response of such vehicles, which reduces the incidence of serious injuries and fatalities in vehicular accidents, provides accurate navigation, introduces parking assistance, and contributes to many other features that reduce human effort and errors, as well.

However, the conventional approaches do not adequately address health and safety of fleet and passenger vehicles where there is a health-related emergency. One in three drivers has a pre-existing condition that makes them more susceptible to having a health event behind the wheel. When a crash is caused by a health emergency, it may be four times as likely to result in a fatality.

### SUMMARY

Conventional solutions lack occupant (e.g., driver, passenger) monitoring software and hardware that detect medical anomalies in the vehicle cabin and coordinate with emergency medical services (EMS) functions to communicate relevant data. Currently, if a driver experiences a medical anomaly in a vehicle, the vehicle would, at best, continue driving autonomously, unaware of the driver's medical event, or, at worst, lead to an accident resulting in injury to the driver as well as to others. In some jurisdictions, drivers prone to such emergencies, such as those with epilepsy and older adults, may face the loss of licensure or other restrictions on vehicle mobility so that the risk of road accidents may be mitigated. Unfortunately, such legal measures may be inconvenient and potentially unfair to many drivers. To tackle this issue, technologies have been attempted in other environments, and none have met a reliable threshold of determining medical anomalies. Some conventional solutions attempt to classify different emergencies, perhaps focusing on only one at first. Current solutions may require a user to initiate an action, for example, push a button in the case of a medical emergency, instead of enabling the vehicle to automatically initiate the emergency response without user intervention.

Vehicle operators can experience conditions relevant to altered mental or physical statuses. The conditions may correspond to one or more medical events (e.g., emergencies) or underlying medical conditions. Various mental statuses may be relevant to vehicle operation. It is desirable to have a solution that addresses the problems above by recognizing an anomaly of a driver and taking an appropriate action. Embodiments described herein attempt to identify emergencies on the road before a vehicle crashes, communicate to the vehicle to proactively avoid the crash, take comfort measures for the driver or passenger experiencing a health emergency, reducing a likelihood of a secondary crash caused by other vehicles on the road, and/or ensure that the driver or passenger in distress receives life-saving care in a timely manner.

Vehicle occupants can experience anomalies such as altered mental or physical statuses responsive to an acute or chronic medical issue. The various mental statuses can be associated with one or more conditions. For example, a condition such as eye closure can correspond to migraine headaches, or a stroke of various severities. According to the systems and methods of the present disclosure, the various conditions may be associated with different actions. For example, an action can include presenting an indication from a user interface indicating that to a user should remain alert, an indication that the user should halt the vehicle, or an activation of an autonomy system of the vehicle to halt the vehicle. Various sensors can detect features of a user such as a facial or body landscape, physiological parameters of a vehicle occupant, or vehicle parameters relating to vehicle control of the vehicle occupant. Based on the features such as the features of a facial landscape, the physiological parameters, or the vehicle parameters, the systems and methods herein can determine a condition of a vehicle occupant, and take an action response to the condition.

At least one aspect is directed to a system. The system can include a data processing system comprising one or more processors coupled with memory. The data processing system can receive sensor data from a plurality of sensors associated with a vehicle, the plurality of sensors comprising a camera and the sensor data comprising video of a vehicle occupant and at least one vehicle occupant physiological parameter. The data processing system can extract a plurality of features of the vehicle occupant from the video. The data processing system can execute a machine learning model using the plurality of features of the vehicle occupant and at least a portion of the sensor data to classify an altered mental status selected from a plurality of altered mental statuses based on at least one condition of the vehicle occupant inferred from the plurality of features and the portion of the sensor data. The data processing system can, responsive to a classification of the altered mental status, generate an instruction to cause the vehicle to perform a navigational action based on the classified altered mental status of the plurality of altered mental statuses of the vehicle occupant.

At least one aspect is directed to a vehicle. The vehicle can include one or more processors coupled with memory. The one or more processors can receive sensor data from a plurality of sensors, the plurality of sensors comprising a camera and the sensor data comprising video of a vehicle occupant and at least one vehicle occupant physiological parameter. The one or more processors can extract, from the video, a plurality of features of the vehicle occupant. The one or more processors can execute a machine learning model using the plurality of features of the vehicle occupant and at least a portion of the sensor data to classify an altered mental status selected from a plurality of altered mental statuses based on at least one condition of the vehicle occupant inferred from the plurality of features and the portion of the sensor data. The one or more processors can convey a message indicative of the altered mental status for presentation by a user interface, responsive to a classification of the altered mental status.

At least one aspect is directed to a method. The method can be performed by a data processing system. The method includes receiving sensor data from a plurality of sensors associated with a vehicle, the plurality of sensors comprising a camera and the sensor data comprising video of a vehicle occupant and at least one vehicle occupant physiological parameter. The method includes extracting from the video, a plurality of features of the vehicle occupant. The method includes executing, by the data processing system, a machine learning model using the plurality of features of the vehicle occupant and at least a portion of the sensor data to classify an altered mental status selected from a plurality of altered mental statuses based on at least one condition of the vehicle occupant inferred from the plurality of features and the portion of the sensor data. The method includes generating an instruction to cause the vehicle to perform a navigational action, responsive to a classification of the altered mental status.

At least one aspect is directed to a system. The system can include a data processing system comprising one or more processors coupled with memory. The data processing system can receive sensor data from a plurality of sensors associated with a vehicle, the plurality of sensors comprising an imaging sensor and the sensor data comprising images or 3D point representations of a vehicle occupant. The plurality of sensors may include a physiological sensor. The plurality of sensors may include the imaging sensor and/or the physiological sensor. The data processing system can extract a plurality of time-series features of the vehicle occupant from the images or 3D point representations. The data processing system can execute a machine learning model using the plurality of time-series features of the vehicle occupant to determine at least one condition of the vehicle occupant inferred from the plurality of time-series features. The data processing system can, responsive to the condition, generate an instruction to cause the vehicle to perform a navigational action.

At least one aspect is directed to a vehicle. The vehicle can include one or more processors coupled with memory. The one or more processors can receive sensor data from a plurality of sensors associated with the vehicle, the plurality of sensors comprising an imaging sensor and the sensor data comprising images or 3D point representations of a vehicle occupant. The one or more processors can extract a plurality of time-series features of the vehicle occupant from the images or 3D point representations. The one or more processors can execute a machine learning model using the plurality of time-series features of the vehicle occupant to determine at least one condition of the vehicle occupant inferred from the plurality of time-series features. The one or more processors can, responsive to the condition, convey a message indicative of the condition for presentation by a user interface.

At least one aspect is directed to a method. The method can be performed by a data processing system. The method includes receiving sensor data from a plurality of sensors associated with a vehicle, the plurality of sensors comprising an imaging sensor and the sensor data comprising images or 3D point representations of a vehicle occupant. The method includes extracting a plurality of time-series features of the vehicle occupant from the images or 3D point representations. The method includes executing a machine learning model using the plurality of time-series features of the vehicle occupant to determine at least one condition of the vehicle occupant inferred from the plurality of time-series features. The method includes generating an instruction to cause the vehicle to perform a navigational action, responsive to the determination of the condition.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts a system including a data processing system, in accordance with some aspects.

FIG. 2 depicts a detailed view of a processing/aggregation circuit, in accordance with some aspects.

FIG. 3 depicts a detailed view of the anomaly detection or classification circuit, in accordance with some aspects.

FIG. 4 depicts a vector space comprising a plurality of classifications, in accordance with some aspects.

FIG. 5 depicts features associated with sensor data received from an imaging sensor, in accordance with some aspects.

FIG. 6 depicts further features associated with sensor data received from an imaging sensor, in accordance with some aspects.

FIGS. 7A, 7B, 7C, and 7D depict features associated with sensor data received from a sensor and a time series representation thereof, in accordance with some aspects.

FIG. 8 depicts features associated with sensor data received from a physiological sensor, in accordance with some aspects.

FIG. 9 depicts a flow chart for a method, in accordance with some aspects.

FIG. 10 depicts another flow chart for a method, in accordance with some aspects.

FIG. 11 depicts a graphical user interface, in accordance with some aspects.

FIGS. 12A, 12B, 12C, and 12D depict example sensor placements and field of views, in accordance with some aspects.

FIG. 13 is a block diagram illustrating an architecture for a computer system that can be employed to implement elements of the systems and methods described and illustrated herein.

### DETAILED DESCRIPTION

Following below are more detailed descriptions of various concepts related to, and implementations of, methods, apparatuses, and systems of anomaly detection. The various concepts introduced above and discussed in greater detail below may be implemented in any of numerous ways.

A data processing system can detect an altered mental or physical status of a vehicle occupant corresponding to a medical anomaly such as a medical emergency, and a gradation thereof, such as a low, medium, or high classification. An altered mental status may depend upon or correlate to a physical status. For example, an altered mental status indicating unconsciousness can correlate to a heart attack; an altered mental status of aphasia or neglect syndrome can correlate to a stroke. The data processing system can monitor data points disposed over a facial landscape of a vehicle occupant, vehicle sensors such as vehicle control sensors and vehicle autonomy sensors, and physiological sensors indicative of a condition of a user. The data processing system can classify a status of an occupant of the vehicle based on the sensor data. For example, the data processing system may include or otherwise employ a classification model or ensemble of classification models. The classification models can be trained to classify a status of an occupant according to one or more predefined statuses, such as statuses relating to a magnitude of an anomaly, an ability to control a vehicle, or a particular medical status. Thus, the data processing system can determine a classification for a status of a user and take an action based on the classified status. An action can include providing an indication corresponding to the classified status (e.g., the classified status itself, medical information associated with the classified status, or an instruction based on the classified status, such as an instruction to seek medical care, pull over, or the like.). An action can include providing an instruction to a decisioning system of the vehicle, such as to halt the vehicle. The action can include conveying an indication of the condition to a provider for the occupant (e.g., convey an indication that the user has undergone a stroke, seizure, or aura migraine.

The disclosed solutions have technical advantages for vehicle control systems. For example, the data processing system can inform a user of an anomaly (such as a mental status) to permit the user to take an action, such as bringing the vehicle to a halt at a desired location, seeking medical care, or so forth. In some embodiments, the data processing system can cause the vehicle to come to a halt responsive to the detected condition, which may reduce a chance of a collision. In the event of a vehicle collision, a time for care correlating to a medical event may be reduced. For example, by identification of the medical condition or severity thereof, a medical provider can be alerted to an underlying condition which may be in need of treatment, such as where a collision is associated with no (or different) injuries.

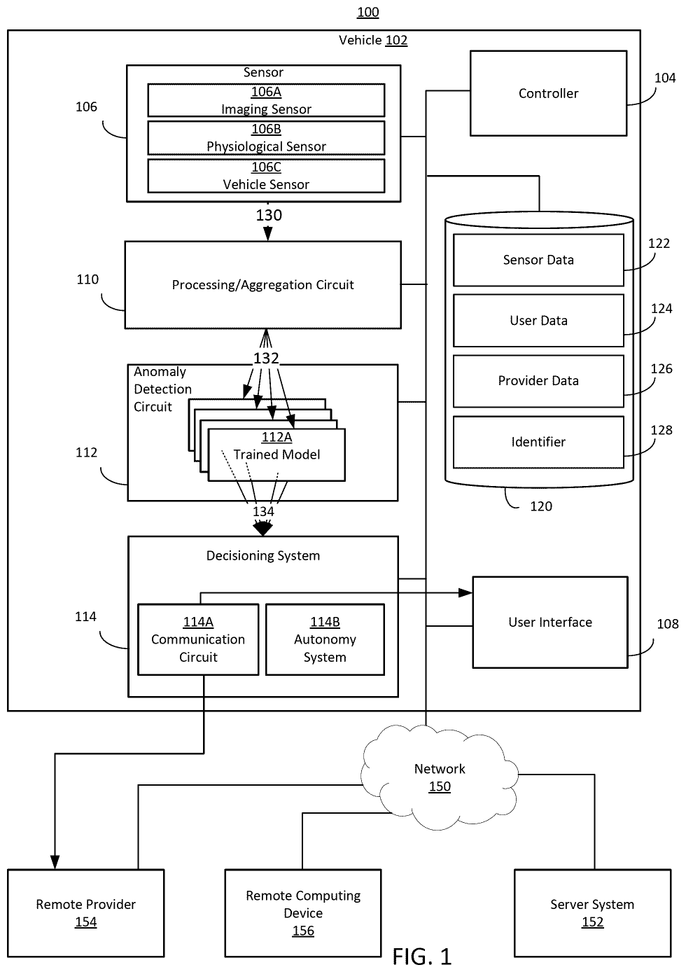

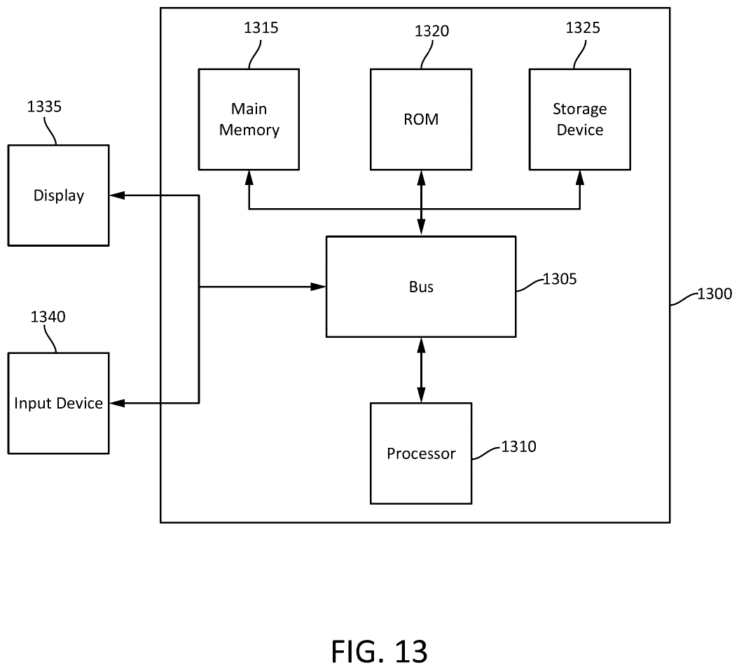

FIG. 1 depicts a system 100 including a vehicle 102, in accordance with some aspects. The vehicle 102 can include or interface with at least one controller 104; sensor 106 including an imaging sensor 106A, physiological sensor 106B, or vehicle sensor 106C; user interface 108; processing/aggregation circuit 110; the anomaly detection or classification circuit 112; decisioning system 114; or data repository 120. The controller 104, sensors 106, user interface 108, processing/aggregation circuit 110, the anomaly detection or classification circuit 112, or decisioning system 114 can each include at least one processing unit or other logic device such as a programmable logic array engine, or module configured to communicate with the data repository 120 or database. The controller 104, sensors 106, user interface 108, processing/aggregation circuit 110, the anomaly detection or classification circuit 112, decisioning system 114, or data repository 120 can be separate components, a single component, or part of the vehicle 102. The vehicle 102 can include hardware elements, such as one or more processors, logic devices, or circuits. For example, the vehicle 102 can include one or more components or structures of functionality of computing devices depicted in FIG. 13. In some embodiments of the present disclosure, the various components of the vehicle 102 can be disposed proximal to each other. In some embodiments of the present disclosure, one or more features of the vehicle 102 can be disposed remote therefrom, and in communicative connection therewith over a network 150.

The network 150 can include computer networks such as the Internet, local, wide, metro, or other area networks, intranets, cellular networks, satellite networks, and other communication networks such as Bluetooth, or data mobile telephone networks. The network 150 can be public or private. The various elements of the system 100 can communicate over the network 150. For example, the vehicle 102 can communicate with one or more server systems 152 and one or more remote providers 154 over the network 150.

Various components of the system 100 can be disposed proximate to the vehicle 102 or remote therefrom. For example, the inclusion of various components proximate to the vehicle 102 may reduce a network load between components of the vehicle 102 and one or more servers 152, 154 over the portion of the network 150. The inclusion of various components remote from the vehicle 102 can decrease network congestion due to model updates, increase available computing capabilities for determining anomalies or classifications, maintain models in a secure data structure or physical location, and so forth. The remote computing device 156 can interface with the vehicle sensors 106, autonomy system 114B, and user interface 108 of the vehicle 102 to detect, classify, or present anomalies. For example, the remote computing device 156 can include one or more instances of a controller 104, processing/aggregation circuit 110, anomaly detection or classification circuit 112, decisioning system 114, or data repository 120. The remote computing device 156 can operate in addition to (e.g., as a part of a voting system, or a shadow system to improve model training) or instead of the components depicted in the vehicle 102. For example, in some embodiments, the vehicle 102 can omit any of a controller 104, processing/aggregation circuit 110, anomaly detection or classification circuit 112, decisioning system 114, or data repository 120 (e.g., one or more data structure stored therein). In some embodiments, feature extraction may be performed locally at the vehicle 102 (e.g., to reduce an amount of data conveyed from the vehicle 102 to the remote computing device 156) and other or subsequent operations can be performed by the remote computing device 156. Operations discussed within reference to the vehicle 102 herein, which are not clearly constrained to the vehicle (e.g., sensor installation location) may also refer to corresponding actions by the remote computing device 156, even where such embodiments are not explicitly described, merely in the interest of clarity and brevity. Either or both of the vehicle 102 components or the remote computing device 156 can be referred to as a data processing system.

The system 100 can include or interface with one or more server systems 152. The server system 152 can refer to or include a cloud computing environment with one or more servers. The server system 152 can receive information from the vehicle 102, or provide information to the vehicle 102. For example, the server system 152 can receive information indicative of a status of the vehicle 102, or an occupant thereof. The server system 152 can associate one or more users with one or more identifiers 128. For example, a first identifier 128 may be unique to a vehicle 102 or an occupant, and a second identifier 128 may refer to an aggregation of unique identifiers 128, or a subset of information from which the server system 152 cannot identify a particular occupant or vehicle 102. The server system 152 can update and covey models, thresholds, and the like to, for example, the processing/aggregation circuit 110, the anomaly detection or classification circuit 112, or the decisioning system 114. For example, the server system 152 can receive information from a plurality of vehicles 102 associated with a unique identifier 128, and determine an update for a model or a threshold based on the information associated with the unique identifier 128. The server system 152 can convey an update to a model or threshold which does not include a unique identifier 128 associated with at least a subset of received indication (e.g., personally identifiable information, such as health information of the sensor data 122).

The system 100 can include or interface with one or more remote providers 154. A remote provider 154 may refer to or include an emergency service provider, a primary medical care provider for the user, an emergency contact associated with a vehicle 102 or user, a data provider associated with a health condition of a user, or the like. The remote provider can include a person who is a non-occupant of the vehicle 102 or a computing device associated therewith.

The data repository 120 can include one or more local or distributed databases, and can include a database management system. The data repository 120 can include computer data storage or memory and can store one or more of sensor data 122, user data 124, provider data 126, or an identifier 128. The sensor data 122 can refer to or include stored data corresponding to various sensors 106 of the data processing system. For example, the sensor data 122 can include temporally longitudinal data such as health data of a user, control data for the automobile, environmental data associated with the automobile, or the like. The sensor data 122 can include salient or processed data for maximum values, minimum values, average values (e.g., rolling time averages), trend lines, the like, or an indication thereof. For example, the sensor data 122 can include longitudinal data, or a representation thereof (e.g., based on features extracted from a data signal) which may be indicative of a historical average of a user or the vehicle 102 (e.g., across multiple trips), or of a current mental status of a user (e.g., across a rolling two-minute average).

A portion of the sensor data 122 that may not be useful for at least one purpose may still be used for other functionality. For example, the sensor data 122 can be corrupt, or otherwise omit useful information (e.g., an image including a glare on eyeglasses may not be useful to determine a direction of gaze, but may be useful for another purpose such as a posture determination). Sensor data 122 that is absent or not useful may be referred to as deficient. Such deficiency can be for one or more purposes. For example, a determination of posture may be determined from sensor data 122 that is deficient with regard to determining a gaze.

The user data 124 can refer to or include information corresponding to one or more vehicle occupants. For example, the user data 124 can include sex, age, weight, height, previous medical history, preferences, and user parameters such as a heart rate, respiration rate, body posture, facial images, or the like. The user parameters can include historical (e.g., baseline) user parameters, or current user parameters. The user data 124 can include prior medical history of an occupant of a vehicle 102.

The provider data 126 can refer to or include information associated with one or more providers associated with the user. The provider data 126 can include contact information such as an electronic address for a location communicatively coupled to the network 150. The provider data 126 can include location information such as a physical or street address which may be navigable by the vehicle 102, such as by a decisioning system 114 thereof. The provider data 126 can include relationship information such as primary care physician, spouse, or available emergency medical technician. The provider data 126 can include information associated with a level of care such as an indication that an individual can provide emergency medical services or routine care.

The identifier 128 can refer to or include an identifier 128 to identify the vehicle 102 or an occupant thereof. For example, the identifier 128 may be or include a unique identifier 128 which associates the user with various user data 124, provider data 126, and the like. A single identifier 128 can correspond to all available information associated with the user or vehicle 102, or various identifiers 128 can be generated for separate portions of the user. For example, an identifier 128 identify a subset of user data 124 which does not personally identify the user. The identifier 128 for the subset of data can include heart rate information, gaze information, or a vector corresponding to a facial image during acceleration exceeding a threshold (e.g., indicative of a collision), an age range, sex, etc., but may omit a precise location, date of birth, etc.

The vehicle 102 can include, interface with, or otherwise utilize at least one controller 104. The controller 104 can include or interface with one or more processors and memory, such as any of the processors described by FIG. 13. The processor can be implemented as a specific purpose processor, an application specific integrated circuit (ASIC), one or more field programmable gate arrays (FPGAs), a group of processing components, or other suitable electronic processing components. The processors and memory can be implemented using one or more devices, such as devices in a client-server implementation. The memory can include one or more devices (e.g., random access memory (RAM), read-only memory (ROM), flash memory, hard disk storage) for storing data and computer code for completing and facilitating the various user or client processes, layers, and modules. The memory can be or include volatile memory or non-volatile memory and may include database components, object code components, script components, or any other type of information structure for supporting the various activities and information structures of the inventive concepts disclosed herein. The memory can be communicably connected to the processor and include computer code or instruction modules for executing one or more processes described herein. The memory can include various circuits, software engines, and/or modules that cause the processor to execute the systems and methods described herein, such as to cause the communication or processing of audio signals.

The controller 104 can include or be coupled with communications electronics. The communications electronics can conduct wired and/or wireless communications. For example, the communications electronics can include one or more wired (e.g., Ethernet, PCIe, or AXI) or wireless transceivers (e.g., a Wi-Fi transceiver, a Bluetooth transceiver, an NFC transceiver, or a cellular transceiver). The controller 104 may be in network communication or otherwise communicatively coupled with the server system, remote provider 154, or other components of the vehicle 102. The controller 104 can cause one or more operations disclosed, such as by employing another element of the data processing system. For example, operations disclosed by other elements of the data processing system may be initiated, scheduled, or otherwise controlled by the controller 104.

The vehicle 102 can include, interface with, or otherwise utilize at least one sensor 106. The sensor 106 can sense or measure a user parameter or a vehicle parameter to generate sensor data 122. The sensors 106 may include sensors configured to collect data regarding a pose or features of the body of a vehicle occupant, and the sensors 106 may include sensors configured to collect data regarding physiological characteristics of the vehicle occupant. In an example embodiment, the sensors 106 may include both an imaging sensor to detect features of the body of the vehicle occupant and sensors to detect physiological characteristics. It is intended, however, that some configurations may include only one type of sensor. Additionally, it is intended that other sensors may be used in addition to those described herein.

The sensors 106 may be configured to collect data and provide the data to another component (e.g., processing/aggregation circuit 110) or device (e.g., remote computing device 156) for processing. In some embodiments, the sensors 106 may be configured to collect data and provide identification of conditions, situations, or classifications based on the data. In other embodiments, the sensors 106 may provide raw or processed data for analysis.

The sensors 106 can include an imaging sensor 106A (e.g., IR or visual spectrum camera, millimeter wave radar, LiDAR, etc.) configured to generate 3D point representation data indicative of various features of a vehicle occupant, such as facial features (e.g., facial droop), body posture, and the like. The sensor may generate a point cloud having a discrete set of data points in space that represent a 3D portion or aspect of the vehicle occupant. In an alternative embodiment, an imaging sensor may generate a 2D image from which features of a vehicle occupant may be extracted. In the example embodiments, 3D point representation data is used to extract features of the vehicle occupant, but it is intended that images may be used instead of or in addition to the 3D point representation data. The one or more imaging sensors 106A can be communicatively coupled to the processing/aggregation circuit 110, to convey the data thereto. The one or more imaging sensors 106A can be positioned to capture 3D point representation data associated with one or more vehicle occupants or portions thereof. For example, a first imaging sensor 106A can be configured to capture 3D point representation data comprising facial landscaping points (e.g., a tip of a nose, chin, eye, and so forth). In some embodiments, the one or more imaging sensors 106A can include various positions or lensing (e.g., fisheye lenses) configured to generate 3D point representation data of a body position. For example, a user may slouch in their seat, be slumped over a steering wheel, holding a steering wheel, or may have limp arms (e.g., as in the case of an unresponsive occupant).

The sensors 106 can include a physiological sensor 106B. A physiological sensor 106B can detect a physiological condition other than facial features or body postures, which may be referred to as a physiological parameter. In some embodiments, the physiological condition can be detected by a same imaging sensor 106A as is employed to generate data indicative occupant features. The physiological sensor 106B can include millimeter wave radar detectors configured to transmit and receive a signal reflected from an occupant, the signal being indicative of a heart rate, breath rate, chest movement, heart rate variability, other vital signs, or the like. The physiological sensor 106B can include thermal imaging sensors or other IR sensors 106, such as sensors 106 configured to measure skin temperature. The physiological sensor 106B can be integral to the vehicle 102 (e.g., in the case of a time of flight sensor 106 mechanically coupled to the vehicle 102 to generate physiological sensor data 122). The physiological sensor 106B can be a user sensor 106 which is communicatively coupled with the vehicle 102. For example, the physiological sensor 106B can be a mobile or wearable device such as a smart watch, chest strap, or glucometer. The physiological sensor 106B can include an electroencephalogram, electrocardiogram, a mobile device including current or previous physiological parameters, or the like. The physiological sensor 106B can be a component of the vehicle 102, or in network communication therewith, such as over an application programming interface (API) corresponding to another device, an Ethernet network or controller area network (CAN) of the vehicle 102, or the like.

The sensors 106 can include a vehicle sensor 106C. The vehicle sensor 106C can include a sensor 106 of a control interface such as a speed sensor 106, steering angle sensor 106, accelerometer, or brake pedal position sensor 106. The vehicle 102 can receive sensor data 122 from a decisioning system 114 such as a distance to another vehicle 102 (e.g., a radar, image, LiDAR, vehicle to vehicle (V2V) system, or the like) from a vehicle sensor 106C. Information contained in sensor data 122 from a vehicle sensor 106C may be referred to as a vehicle parameter. The sensor data 122 may be conveyed to, for example, the processing/aggregation circuit 110 as depicted by a first data flow path 130.

The vehicle 102 can include, interface with, or otherwise utilize at least one user interface 108 to exchange information with an occupant of the vehicle 102 or other person or entity. The user interface 108 can include an input device. The input device can include various knobs, button, display screens (e.g., touchscreens), indicator lights, or the like, which are disposed within the vehicle 102. For example, the input device can include vehicle controls such as steering wheels, throttle pedals, and so forth. The user interface 108 can include a graphical user interface 108 such as a graphical user interface instantiated by a display of the vehicle 102, or another device in network communication with the vehicle 102. For example, the user interface 108 can include a mobile phone, laptop or desktop computer, augmented reality (AR) or virtual reality (VR) device, or another network connected device of a provider associated with the vehicle 102 or an occupant thereof.

The user interface 108 can present information (e.g., a user parameter or vehicle parameter). For example, the user interface 108 can provide an operator or other occupant of a vehicle 102 or another person remote therefrom, a notification of a detected condition of the driver. The user interface 108 can present a prompt for information, confirmation, or other input. For example, the user interface 108 can present a prompt for the user to confirm that they are aware of the information, that they are alert, whether they wish for a decisioning system 114 of the vehicle 102 to bring the vehicle 102 to a halt, whether they wish to summon medical services, or the like. The user interface 108 can provide an indication to proceed to a medical provider location such as a physical address, responsive to a condition of the user.

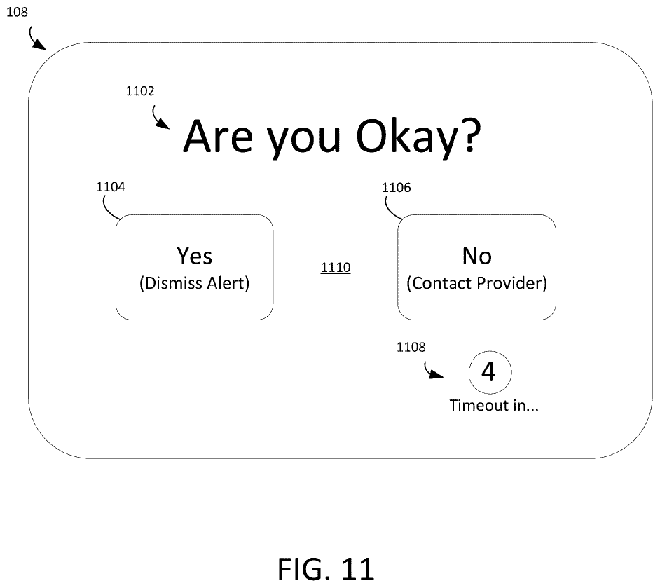

The user interface 108 can receive inputs. An example user interface is illustrated in FIG. 11 and described in more detail below. For example, a user can input various preferences, user data 124, throttle pedal positions, steering angles, or the like via the user interface 108. The inputs can include responses to prompts. The responses can include explicit user inputs (e.g., a press of a button, a spoken command to a voice system, or a motion, gesture, or the like). The responses can include a time-out input. For example, the user interface 108 can determine that a lack of an explicit user input corresponds to response to a question (e.g., a lack of response to “do you require medical assistance” may be indicative of a response in the affirmative). The user interface 108 can present a prompt or other indication to a provider such as a caretaker or emergency medical personnel. For example, the indication can provide the classification or request information such as if medication is available, was taken earlier in a day, or so forth.

The user interface 108 can receive input associated with a classification such as a classification of an anomaly such as a classification between an anomalous or non-anomalous event, a classification of a severity of an anomaly, or of a particular medical event. The user interface 108 can provide a prompt for a user to select, such as to confirm their alertness, or symptoms. For example, the user interface 108 can present a question inquiring as to particular symptoms of the user, or providing an option for a user to override the selection. The user interface 108 can provide a prompt for a maskable condition or mental status (e.g., a condition which is determined with low confidence, or a condition which may not be associated with an immediate action). Based on the response (e.g., a delay prior to receiving the response, or a content of the response itself), the data processing system can adjust a response or a confidence. For example, if a user response to a prompt associated with mental confusion is immediate and selects a negative response, the confidence may be lowered. If a user response to mental confusion is delayed, and includes dragging fingers across a touchscreen outside of a response area, a confidence level may be raised. Some conditions such as altered mental statuses may not correspond to a prompt (e.g., a condition may not be maskable). For example, a condition indicative of an unconscious user may omit a prompt. In some embodiments, the user interface 108 can convey an indication of a classification which may or may not identify the particular classification. (e.g., “you may be experiencing a stroke, please halt the vehicle” or “you may be experiencing a medical emergency, autonomy system engaged”).

The vehicle 102 can include, interface with, or otherwise utilize at least one processing/aggregation circuit 110 configured to extract a user parameter or vehicle parameter from the sensor data 122. The processing/aggregation circuit 110 can generate features from one or more sensors 106. The features can include heart rates, breath rates, blood oxygenation levels, eye gaze or other facial features, which may be indicative of a condition associated with a condition (e.g., eye droop, face droop, wincing, distress, or slackened facial muscles). In various embodiments, the features can include longitudinal data or features thereof, including, a trend line, rolling average, maximum, minimum, or other values further derived from other features. In various embodiments, the anomaly detection or classification circuit 112 can determine or classify an anomaly based on the non-longitudinal data. For example, in a first embodiment, a processing/aggregation circuit 110 can extract longitudinal data (e.g., heartrate), and extract a longitudinal feature (e.g., rapid onset increased heart rate) therefrom. The processing/aggregation circuit 110 can convey the longitudinal feature for input to the anomaly detection or classification circuit 112. In a second embodiment, a processing/aggregation circuit 110 can determine longitudinal data (e.g., the heartrate) for input to the anomaly detection or classification circuit 112, whereupon the anomaly detection or classification circuit 112 may include a recursive neural net or other time-relevant model (e.g., long short-term memory (LSTM) or gated recurrent unit (GRU) RNN, temporal convolutional net (TCN), transformer, the like, or combinations thereof).

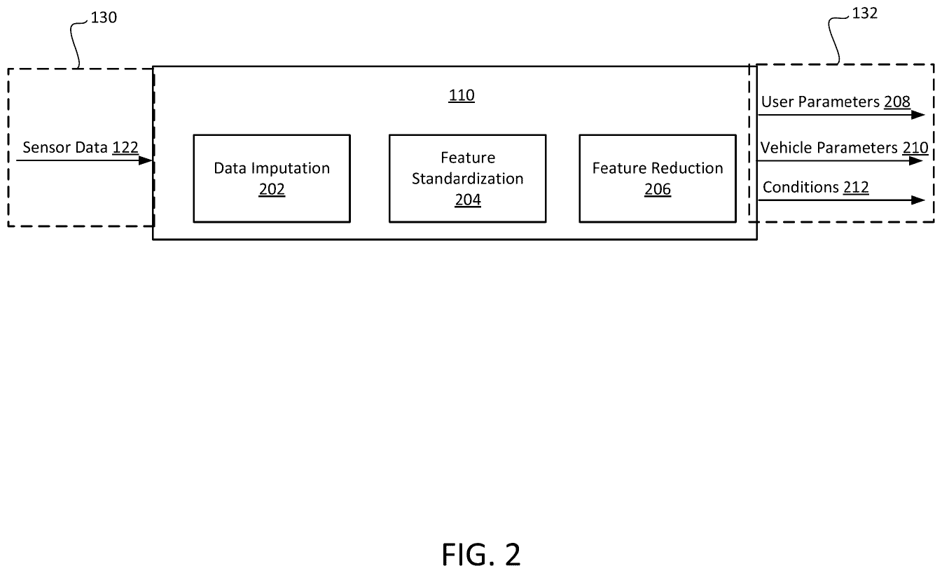

The processing/aggregation circuit 110 can organize, align, interpolate, truncate, flatten, tag or otherwise process sensor data 122 for ingestion by trained models 112A of the anomaly detection or classification circuit 112. For example, the processing/aggregation circuit 110 can receive sensor data 122 from a plurality of sensors 106. The sensor data 122 can include image data from an imaging sensor 106A (e.g., video data of a vehicle occupant such as a vehicle operator) physiological data (e.g., a physiological parameter of a vehicle operator). The processing/aggregation circuit 110 can extract various features of the vehicle occupant, such as face landscape points. The processing/aggregation circuit 110 can generate an input for the anomaly detection or classification circuit 112 comprising features from the plurality of sensors 106. In some embodiments, the processing/aggregation circuit 110 can detect a condition associated with a user. For example, a condition can be based on one or more features. For example, a condition may include tachycardia, hypoglycemia, arrhythmia, or the like. As depicted, the processing/aggregation circuit 110 can convey an output thereof to the anomaly detection or classification circuit 112 along a second data flow path 132. The processing/aggregation circuit 110, including such a second data flow path 132, is further described with regard to FIG. 2.

The vehicle 102 can include at least one anomaly detection or classification circuit 112. The anomaly detection or classification circuit 112 can determine a presence or type of anomaly. The anomaly detection or classification circuit 112 may use one or more of various methods for identifying an anomaly. In one configuration, the anomaly detection or classification circuit 112 may use an unsupervised approach whereby it is trained with data representing normal driving behavior to identify unusual behavior. The anomaly detection or classification circuit 112 can also identify anomalous non-emergency behavior. In this configuration, the anomaly detection or classification circuit may not output a confidence level, but rather would provide a loss using a dynamic threshold to classify an anomaly versus normal situation. In another configuration, the anomaly or classification circuit 112 may use a supervised approach to provide a binary classification of an emergency versus non-emergency situation. In another configuration, the anomaly or classification circuit 112 may use an n-classification approach that do not classify particular health emergencies (e.g., may not diagnose a heart condition as a heart attack), but will classify a level of emergency using a plurality of levels, such as but not limited to: non-emergency, mild emergency, medium emergency, major emergency, or critical emergency. As a result, the anomaly detection or classification system 112A can be configured to perform a detection and/or classification of an anomaly situation.

In one example, trained models 112A of the anomaly detection or classification circuit 112 can include one or more machine learning models (e.g., an ensemble of machine learning models) configured to classify a condition such as an altered mental status from a plurality of altered mental statuses, a severity of an anomaly from a plurality of severities, or a binary classification indicative of a presence of an anomaly. Some conditions may be determined by either of the processing/aggregation circuit 110 or the anomaly detection or classification circuit 112. For example, hypoglycemia can be determined by the processing/aggregation circuit 110 based on a glucose monitor, or by the trained models 112A of the anomaly detection or classification circuit 112 based on a skin temperature sensor 106, change in heart rate, and change in focus, or the trained models 112A of the anomaly detection or classification circuit 112 can determine a condition severity based on the condition without an explicit determination of a particular medical condition. The trained models 112A of the anomaly detection or classification circuit 112 can classify the condition based on the information received from the processing/aggregation circuit 110. For example, the trained models 112A of the anomaly detection or classification circuit 112 can receive an input set of features associated with a plurality of sensors 106. The input set can be received according to a normalized input data structure. For example, the input set can include a data structure including information from a plurality of sensors 106, including features from data along with physiological features from one or more physiological sensors 106.

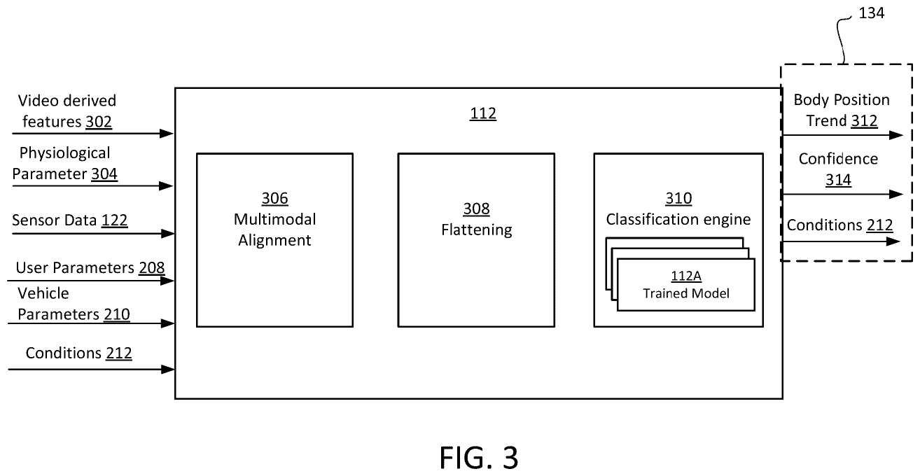

The anomaly detection or classification circuit 112 can classify a condition or severity thereof according to one or more conditions or severities. For example, the one or more conditions can correspond to a location of a vector space or a Boolean indication. As depicted, the anomaly detection or classification circuit 112 can convey an output thereof to the decisioning system 114 along a third data flow path 134. The anomaly detection or classification circuit 112, including such a third data flow path 134, is further described with regard to FIG. 3.

The vehicle 102 can include at least one decisioning system 114. The decisioning system 114 can control the vehicle 102 by one or more vehicle controls which may be a same user control actuated by a user, or another control. An autonomy system 114B of the decisioning system 114 can execute navigational actions. For example, the decisioning system 114 can engage a braking system to bring a vehicle 102 to a halt, a steering system to control a vehicle direction such as to cause the vehicle 102 to exit a roadway in coordination with braking. The autonomy system 114B of the decisioning system 114 can cause the vehicle 102 to perform an autonomous action based on a classification of condition such as an altered mental status of a user. For example, the autonomy system 114B can cause the vehicle 102 to navigate to a physical location of a medical provider. The decisioning system 114 can execute non-navigational actions. For example, the decisioning system 114 can cause an audial or visual alert for an occupant of the vehicle or other drivers. For example, the decisioning system 114 can engage a vehicle horn, chime, or hazard warning lights responsive to the classification of the altered mental status, such as via network communication with the user interface 108. The engagement of the audial or visual alert can vary according to a condition. For example, a first condition severity may not be indicative of halting the vehicle 102, whereupon the vehicle 102 can engage hazard warning lights. A second condition severity may be indicative of halting the vehicle 102, whereupon the vehicle 102 can engage hazard warning lights along with sounding a horn.

A communications circuit 114A of the decisioning system 114 can communicate with a remote provider 154 or the user interface 108 (e.g., disposed within or remote from the vehicle 102). The communications circuit 114A can include various transceivers such as network interface transceivers. The decisioning system 114 can convey the various messages described herein. For example, various messages or actions associated with a classification determined by the trained models 112A of the anomaly detection or classification circuit 112 can be conveyed by the decisioning system 114, via the communications circuit 114A. Moreover, any look up tables, thresholds, or other decisioning logic can refer of the decisioning system 114.

FIG. 2 depicts a detailed view of a processing/aggregation circuit 110, in accordance with some aspects. The processing/aggregation circuit 110 can ingest sensor data 122 from the one or more sensors 106 including the imaging sensor 106A, physiological sensor 106B, or vehicle sensor 106C, along the first data flow path 130 depicted in FIG. 1. The sensor data 122 associated with each sensor 106 may be associated with a different time, content, sample rate, or the like. For example, video data may be provided at a 60 frame per second or 1 frame per second period (e.g., as a series of images or series of 3D point representations) whereas other data such as millimeter wave radar can be received at a period in a microsecond or millisecond range. Although the example embodiment may recite the use of time-series data to determine a condition, it is intended that other features, including non-temporal features, may be used instead of or in addition to the time-series data. The non-temporal features may be extracted from one or more images, frames, or 3D point representation data, or may be extracted from other non-image sensor data.

A data imputation engine 202 can sparsify received sensor data 122. For example, the data imputation engine 202 can subsample an input of sensor data 122, or can determine a representative sample for a series of data samples. For example, a representative sample can be or include a first, last, median, maximum, minimum, smoothed, or other derivative of the sensor data 122. Likewise, a data imputation engine 202 can densify received sensor data 122. For example, the data imputation engine 202 can define data according to linear, polynomial (e.g., spline), or other interpolation. For example, a pattern such as breathing, heartrate, or the like can be interpolated according to a portion thereof (e.g., missing data in a QRS complex may be substituted for an average of other QRS complexes). The data imputation engine 202 can determine a quantity of deficient sensor data 122, compare the quantity of deficient sensor data 122 to a threshold, and impute sensor data 122 responsive to the quantity of deficient sensor data 122 being less than a threshold. In some embodiments, the data imputation engine 202 can tag a portion of sensor data 122 as deficient, or clear a portion of the data. For example, the data imputation engine 202 can indicate one or more nonce values for deficient sensor data 122, such that a machine learning model can disregard or employ the existence of the deficient sensor data 122.

The feature standardization engine 204 can align two-dimensional imaging sensor data with three-dimensional occupants or environments. For example, the feature standardization engine 204 can adjust a skew or tilt of a head position to determine a three-dimensional location of features of a facial landscape, or infer a two dimensional location of a facial landscape in a predefined orientation (e.g., a straight on image). The feature standardization engine 204 can adjust a height of an occupant or other feature, such as an inter-pupillary distance, cheek to cheek distance, or the like. The feature standardization engine 204 can operate in a calibration mode during a first period of operation to determine the one or more adjustment, and thereafter extract features according to the one or more adjustments. In some embodiments, the feature standardization engine 204 can recalibrate during operation. For example, the feature standardization engine 204 can recalibrate periodically or in response to a deviation of a position of a user which exceeds a threshold. In some embodiments, the feature standardization engine 204 can calibrate simultaneous to operation. For example, a running average of a position may be employed to adjust a center point for a position.

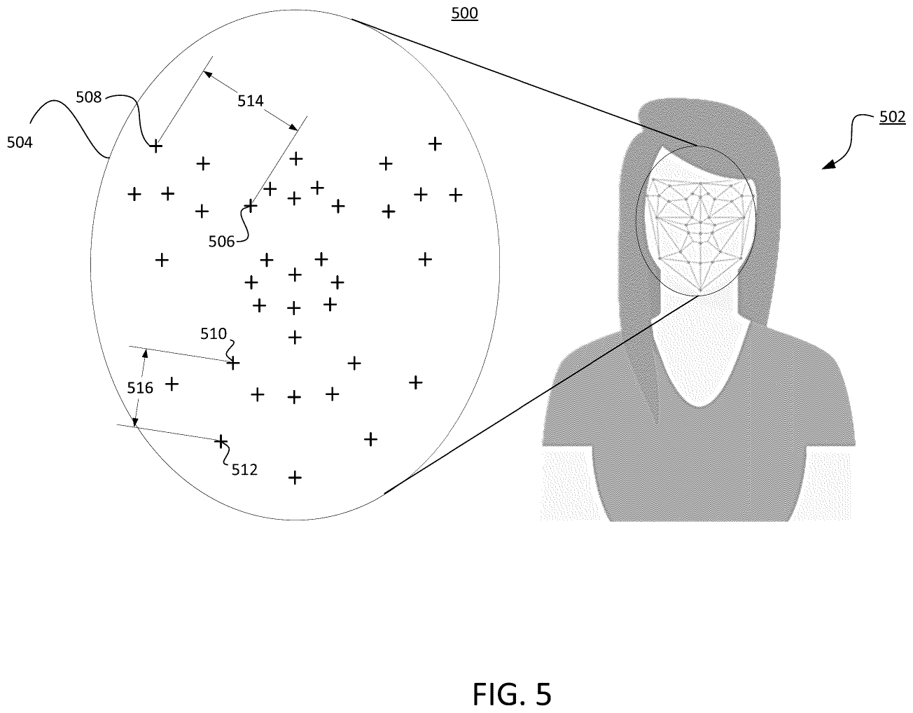

The feature standardization engine 204 can standardize sensor data 122 received from a vehicle sensor 106C (e.g., an average or maximum steering angle sensor turn rate, throttle pedal adjustment rate, or the like). For example, the feature standardization engine 204 can standardize inputs on one or more of a vehicle basis, occupant basis, or session basis. The standardization can be based on a z score or other statistical representation of data variation. The standardization can further include omission or flagging of outliers. An example of standardization for a facial landscape is provided henceforth, at FIG. 5.

The feature standardization engine 204 can standardize sensor data 122 received from a physiological sensor 106B. For example, a heart rate, gaze, or the like can be adjusted based on a particular user or other user data 124 such as an age, height, weight, sex, or the like. In one example, a heartrate can be adjusted based on historical heart rate information for the user, or based on an expected heart rate for the person based on a height, weight, or other medical information. The feature standardization engine 204 can standardize the data by adjusting the sensor data 122 or a threshold associated therewith. For example, data can be input to the anomaly detection or classification circuit 112 according to a predefined input range (corresponding to a machine learning model of the trained models 112A of the anomaly detection or classification circuit 112).

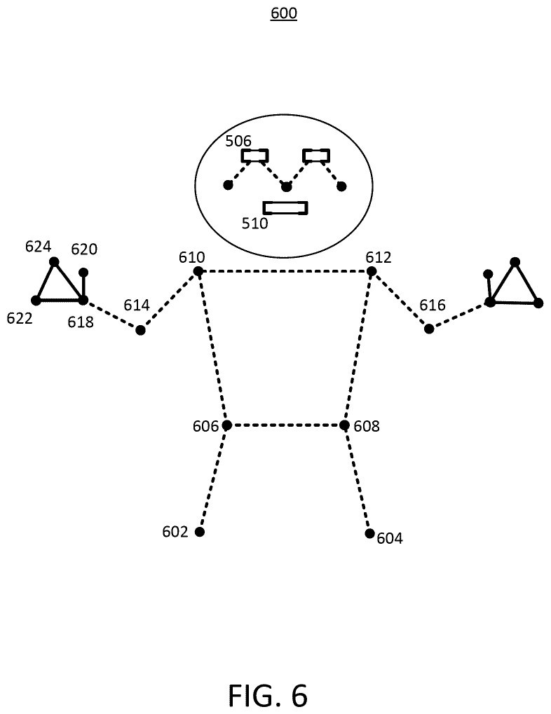

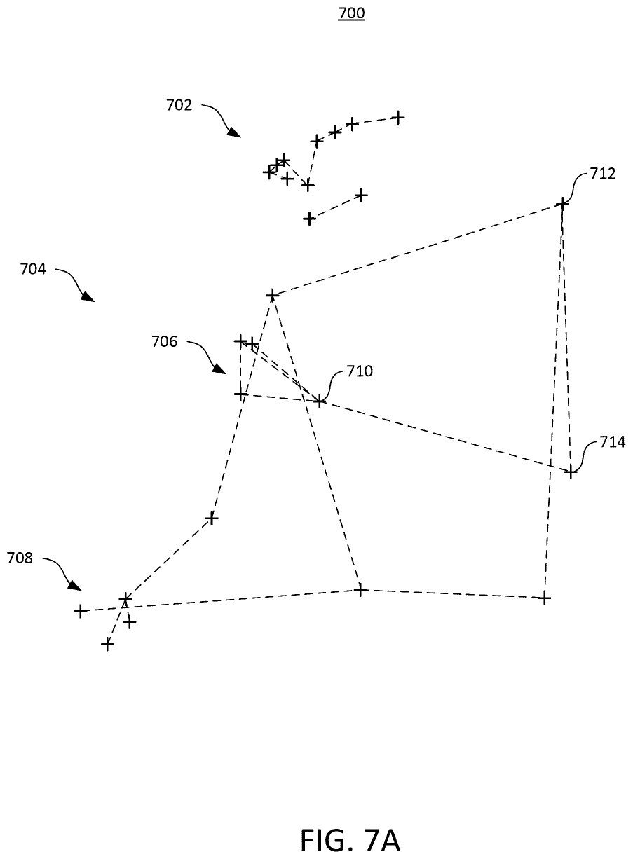

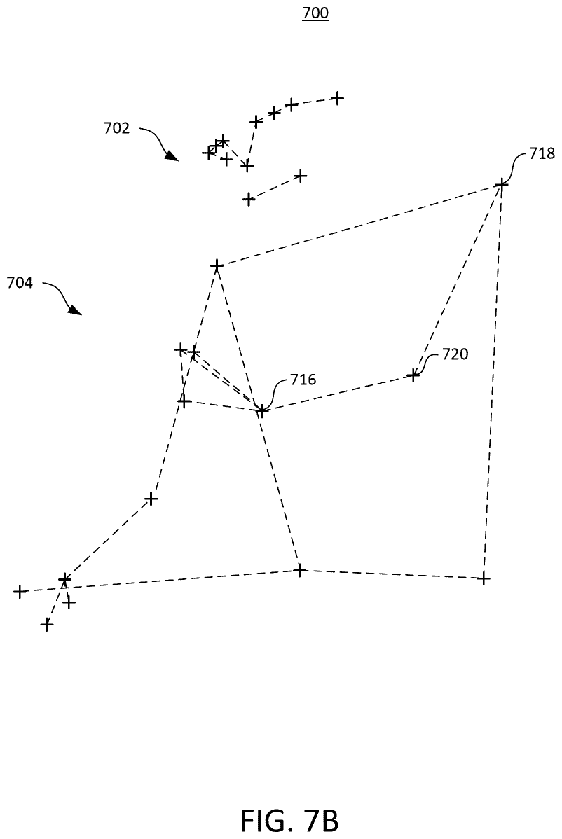

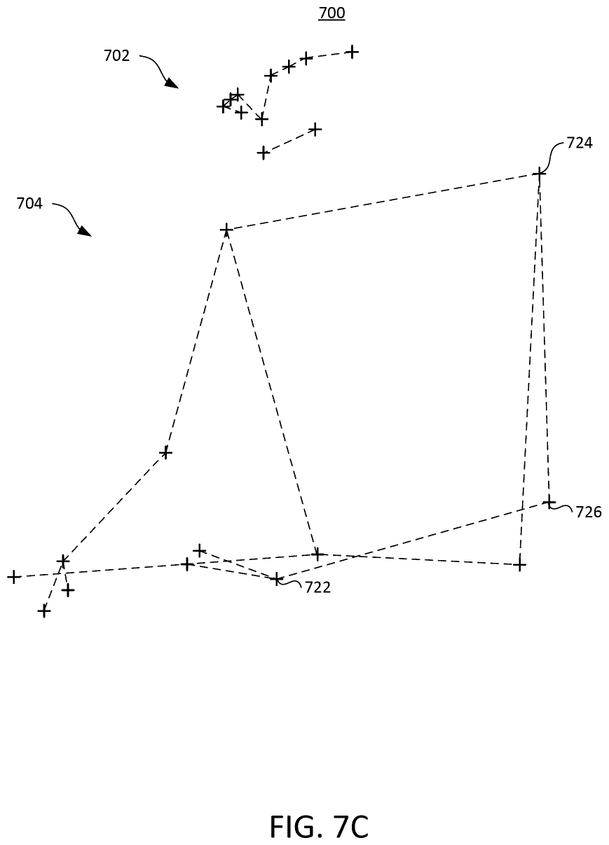

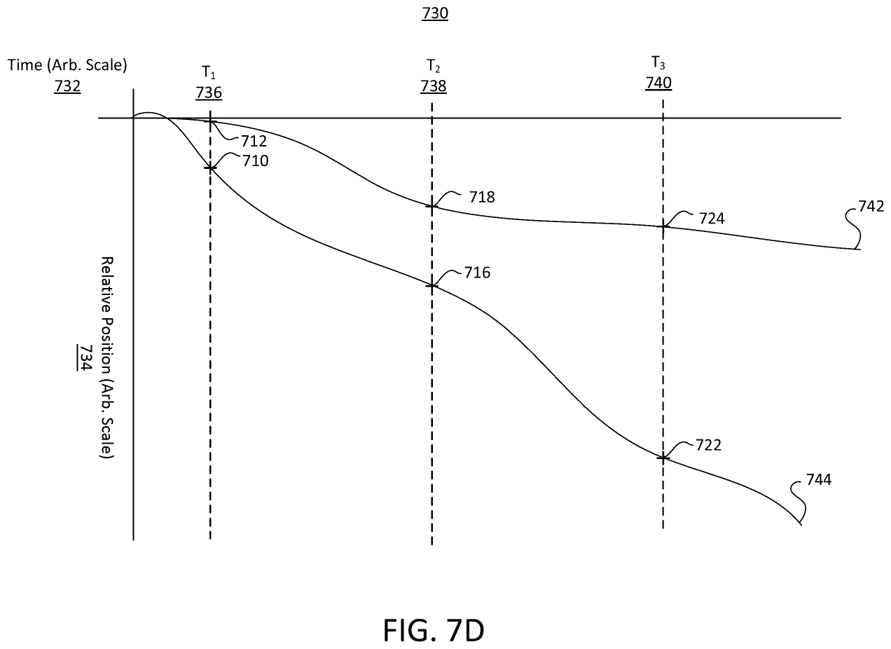

The processing/aggregation circuit 110 can include a feature reduction engine 206. The feature reduction engine 206 can determine responses from the various sensor data 122. An example of feature reduction for 3D point representation data of a facial landscape is provided henceforth at FIG. 5. An example of feature reduction for 3D point representation data of a body position is provided henceforth at FIG. 6. An example of feature reduction for a heart rate is provided at FIG. 7. The feature reduction can form a flat (e.g., one or two dimensional) data structure. The feature reduction can generate a data structure corresponding to one point in time. The feature reduction engine 206 can generate a data structure corresponding to multiple points in time. A data structure conveyed from the processing/aggregation circuit 110 to anomaly detection or classification circuit 112 can include overlapping data (e.g., overlapping windows), or data provided in a sequence configured for receipt by a time-sensitive trained models 112A of the anomaly detection or classification circuit 112 (e.g., an RNN).

The feature reduction engine 206 can generate user parameters 208 comprising reduced features such corresponding to heart rate, vehicle parameters 210, or the like. For example, the reduced features can include a Boolean indication of a departure from a normal range. The reduced features can include an indication of one or more predefined statuses (e.g., slightly elevated, moderately elevate, or severely elevated). The reduced features can include a numeric indication of a heart rate from a time-series indication of voltage levels. The feature reduction engine 206 can generate normalized data from a variety of data sources. For example, various mobile devices, chest straps, or other devices in network communication with the vehicle 102 (e.g., via an API) may include various data formats, which can be adjusted to interface the anomaly detection or classification circuit 112. The processing/aggregation circuit 110 can normalize various user data 124 or provider data 126. For example, the processing/aggregation circuit 110 can provide a normalized indication (e.g., according to a predefined value) for a familial history, diagnosis of a chronic condition, historical diagnosis, or other information to the anomaly detection or classification circuit 112.

The feature reduction engine 206 can generate vehicle parameters 210 such as a reaction time of the vehicle controls, a distance between the vehicle and a lane marking, or a vehicle path relative to a gaze of an occupant. The vehicle parameters 210 can include current parameters, average parameters, over time, or the like. A vehicle parameter 210 can include a change in speed or direction, which may correlate with an attention of a user. For example, a sudden application or removal of throttle may be conveyed to the anomaly detection or classification circuit 112, along with a classification of a condition 212 of a driver in the same time period (e.g., eyes closed, slumped over the steering wheel).

The feature reduction engine 206 can determine one or more conditions 212 of a user. For example, a user condition 212 may include an elevated heartrate, fever, eye closure, or so forth. The condition 212 may be correlated with one or more conditions. For example, an elevated heart rate can be associated with diabetic ketoacidosis, early phase hypoglycemia, sepsis, thyroid storm, or stroke; face droop may be associated with stroke. A condition may be relevant to control of a vehicle 102, and medical needs of an occupant thereof (e.g., which may be indicative of unsafe operation). The outputs may be conveyed along the second data flow path 132 depicted in FIG. 1.

FIG. 3 depicts a detailed view of an anomaly detection or classification circuit 112, in accordance with some aspects. In various embodiments, other classification circuits 112 can detect or classify other anomalies, conditions or severities thereof. The anomaly detection or classification circuit 112 can receive features extracted from videos (e.g., a plurality of still frames in a sequence). The anomaly detection or classification circuit 112 can include one or more classification models to classify a condition (e.g., a condition indicated by an altered mental status or body position trend 312) from a set of various predefined body position trends 312 (based on time-series data) associated with medical events indicative of an inability to control a vehicle, or other conditions. The anomaly detection or classification circuit 112 can receive multi-modal data. For example, the trained models 112A of the anomaly detection or classification circuit 112 can receive video derived features 302, physiological parameters 304, user parameters 208, vehicle parameters 210, identified conditions 212, or other sensor data 122 or derivatives thereof. The anomaly detection or classification circuit 112 can include a multimodal alignment engine 306. The multimodal alignment engine 306 can align features according to a vehicle occupant, physical dimension, user data 124, or the like. For example, the multimodal alignment engine 306 can temporally adjust a position of one or more portions of sensors data 122, such as by adjusting data for sensors 106 having different latency, to align discrete and continuous transmitters, and so forth. The alignment can be based on prior or known sensor latency, or one or more alignment data (e.g., a tone or light generated by the vehicle 102). The alignment can be based on information derived from various inputs. For example, a vehicle parameter 210 can be aligned to a body position based on an observable steering control of the user (e.g., turning a steering wheel) such that an association between the user parameter 208 and the vehicle parameter 210 correlates the respective sensor data 122.

A flattening engine 308 can align the various sensor data 122 into a predefined data structure. For example, the flattening engine 308 can align sensor data 122 from various sources into the predefined data structure, or can adjust positions within a data structurer based on temporal locations thereof. For example, the flattening engine 308 can move data between data structures which are associated with a single point in time or adjust a location within a data structure that spans a time interval.

A classification engine 310 can refer to or include a time-relevant model such as a Recurrent Neural Network (RNN). The RNN can input a data structure including data for a time interval. The RNN can maintain a hidden state acting as a form of memory between an input and output state, allowing information to persist between iterations. The RNN can be trained on instances of the one or more predefined altered mental statuses, body position trends 312, or other data such as transitions between an unaltered mental status and the altered mental status. The RNN can indicate a correlation (e.g., a prediction of a body position trend 312) of inputs which correspond to outputs indicative of the condition 212 or status that the RNN was trained on. For example, the inputs may refer to sequential data, where temporal dependencies play a role in classification, such as a transition of facial features between positions, a change in heart rate, or so forth. An example of time-series data associated with a body position trend 312 is provided with regard to FIGS. 7A, 7B, 7C, and 7D.

The classification engine 310 can refer to or include a support vector machine (SVM) model to classify a mental status. The SVM can place a vectorized representation of the data structure into a multidimensional plane including various locations or clusters corresponding to various potential events. The SVM model can determine a line or hyperplane separating a first set of potential events from a second set of potential events. The vector corresponding to a predicted body position trend 312 may be disposed on a first or second side of the line or hyperplane. The SVM model can employ multiple iterations until only one event of the set of potential events is on the selected side of a hyperplane. The SVM model can include more than one line or hyper plane to define a volume of the vector space associated with the various predefined events. Upon determining the most likely body position trend 312, the classification engine 310 can determine a difference between the most likely body position trend 312 and the data structure, to determine a confidence level 314 of the most likely altered mental status, or employ another indication of binary classification between the most likely body position trend 312 and non-anomalous data. In some embodiments, a confidence level 314 is not conveyed from the classification engine 310. For example, confidence can be a binary confidence based on a reporting or non-reporting of the body position trend 312 or other condition.

The classification engine 310 can refer to or include a random forest model that receives the data structure. The random forest model can include any number of individual decision trees (e.g., about 200 decision trees). The branches for the trees can be based on, for example, the known relationships described above. The decision trees can include a minimum or maximum number of layers for the individual decision trees (e.g., between two and ten layers). According to some embodiments, the branches may be determined by various techniques such as information gain or Chi-square techniques. A confidence level 314 of the random forest model can depend on a confidence 314 of the random forest (e.g., a number of decision trees selecting a domain of the domain set). In some embodiments, the confidence level 314 of the random forest model can depend on a correct classification rate of test data (e.g., out-of-bag data to test individual decision trees, or separate test data not used to train any decision trees of the random forest).

The random forest model can output one or more predicted events, and may determine a confidence level 314 for each prediction, such as according to a number or attribute of trees sharing a same classification. For example, a random forest wherein 150 of 200 trees predict a particular classification may correlate to higher confidence level 314 than a random forest wherein 100 of 200 trees predict the particular classification.

In various embodiments an ensemble of various machine learning models may determine a classification for a body position trend 312. For example, the ensemble model can include voting between the models. The ensemble model can include weighting between the models (e.g., according to a confidence level 314 or predictive power of each of the models). The ensemble model can include a cascade model such as an SVM to determine a most likely body position trend 312 and a subsequent other model to act as a binary classifier to determine a presence or confidence level 314 of the body position trend 312 relative to an un-altered mental status. The outputs may be conveyed along the third data flow path 134 depicted in FIG. 1.

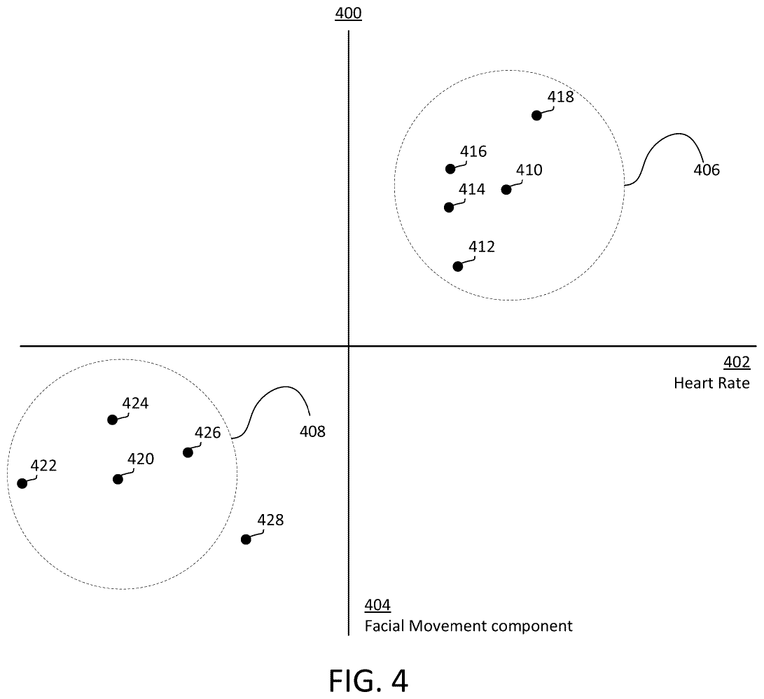

FIG. 4 depicts a vector space 400 comprising a plurality of classifications, in accordance with some aspects. Although the vector space 400 is depicted in two dimensions, merely for ease of depiction, various embodiments can include several dimensions (e.g., thousands of dimensions). A first depicted dimension 402 can correspond to a heart rate. For example, the dimension 402 can correspond to a numeric indication of a heart rate, a change in heart rate from a baseline heart rate, a trend line of a heart rate, or a ratio between a heart rate and other sensor data 122, such as a vehicle speed or a pupil dilation. A second depicted dimension 404 can correspond to a facial movement component such as a location of a feature of a facial landscape. The facial movement can be associated with a distance between points, a derivative of such a distance, or the like.

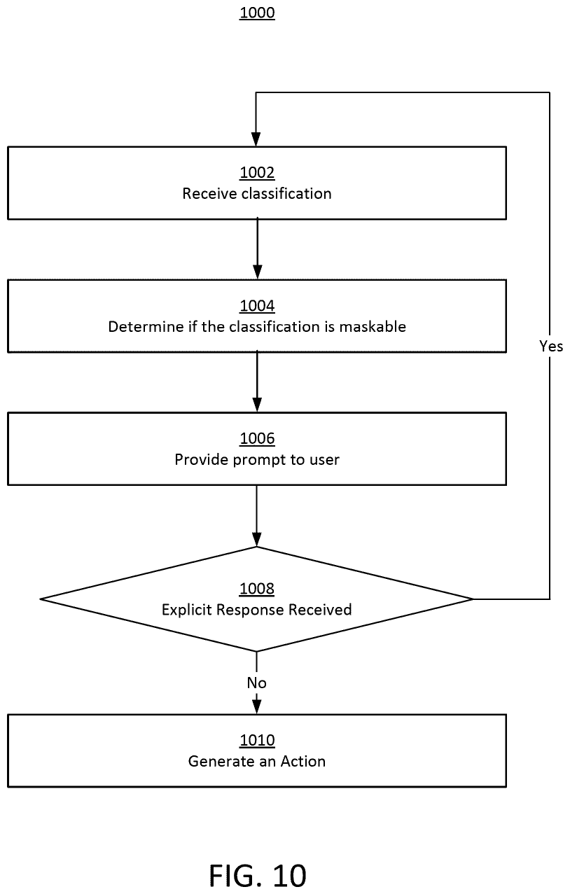

The vector space 400 can include a first cluster 406 and a second cluster 408 corresponding to a first and second condition associated with different body position trends 312, respectively, or a severity thereof. The first cluster 406 can include a first centroid 410 along with a first 412, second 414, third 416, and fourth 418 event. The events can be indicated, received, predicted (e.g., synthesized) by the sever system 152, such that the first cluster 406 is defined by the sever system 152 based on the events and provided to the vehicle 102 (e.g., omitting the events). Likewise, the second cluster 408 can correspond to a second centroid 420 along with a fifth 422, sixth 424, seventh 426, and eighth 428 event. Some events, such as the depicted eighth event 428 may not be bounded by a boundary corresponding to the second cluster 408. The eighth event 428 may be associated with the second cluster 408 by another metric such as a distance to the second centroid 420 or to other events corresponding to the second cluster 408 of the events. The distance to the boundary, the centroid 420, or to other events may be indicative of a confidence level 314 of a classification. For example, a boundary may correspond to a threshold to take an action. In response to the distance between the eighth event 428 and the boundary, the systems and methods of the present disclosure can take an action such as providing a prompt for input from an occupant or non-occupant to adjust a confidence level 314 as is further described with regard to the method 1000 of FIG. 10.

The vector space 400 is not intended to be limiting. Various embodiments may employ different techniques to classify a model or determine a classification thereof. For example, as indicated above, a number of decision trees indicating an output, a response to a prompt, or other data may be employed to classify, verify, or adjust a confidence level 314.

FIG. 5 depicts a feature set 500 associated with sensor data 122 received from an imaging sensor 106A, in accordance with some aspects. As depicted, a facial landscape of an occupant 502 is captured by the imaging sensor 106A. For example, features such as a distance 514 between an eye center point 506 and a point disposed along a hairline 508, a distance 516 between an edge of a mouth 510 and a jawline 512, or so forth. The various features can be mapped onto a data map 504. For example, the data map can be a normalized data map 504 for distance between various features which may be normalized to a head size, inter pupillary distance, or other attribute of an occupant.

FIG. 6 depicts a further feature set 600 associated with sensor data 122 received from an imaging sensor 106A, in accordance with some aspects. A subset of the depicted features can overlap with or correspond to the features of FIG. 5. For example, the feature set 600 can include an eye position 506 or mouth position 510 which is a same position as depicted in the feature set 500 of FIG. 5, or is offset therefrom by a known distance. The data processing systems can employ the overlapping or corresponding positions to temporally align information of the imaging sensor 106A associated with FIG. 5 with the imaging sensor 106A associated with FIG. 6. Further overlapping or correlated data can be employed to align further sensor data 122. The overlapping or corresponding data can be employed to densify data. For example, upon a vehicle occupant moving their face out of frame, the data processing system can employ information from the further feature set 600 to interpolate features from the feature set 500 of FIG. 5.

The depicted feature set 600 can include additional information that may be indicative of a body posture which is visible to an imaging sensor 106A. For example, the imaging sensor 106A can observe a leg position feature 602, 604, waist position feature 606, 608, shoulder position feature 610, 612, and so forth. The body position can further include an arm position feature 614, 616, various hand position features, such as a feature corresponding to a position of a wrist 618, thumb 620 and terminal hand feature 622 and complementary terminal hand feature 624 for a right hand, and similar features of a left hand. Indeed, various features may be disposed symmetrically over a body or face which may improve detection of various altered mental statuses such as stroke based on a detected body position trends 312 such as a progression of asymmetry between left and right-side features.

FIGS. 7A, 7B, 7C, and 7D depict features associated with sensor data received from a sensor 106 and a time series representation thereof, in accordance with some aspects. For example, the features can be features associated with an imaging sensor 106A and observed or determined from a frame, image, or 3D point representation from the imaging sensor 106A. The time-series data can be indicative of an anomaly or medical emergency, such as stroke. Referring now to FIG. 7A, features of a first time, T₁, of a time-series feature set 700 includes facial features 702 and body position features 704 from a first 3D point representation in a sequence from the imaging sensor 106A. The body position features 704 include, for example, the feature set 600 of FIG. 6. Particularly, body features of a left arm 706 and right arm 708 are depicted, such that asymmetries therebetween can be detected. In further detail, a time-series feature set 700 can include a left wrist feature, left elbow feature, and left shoulder feature, as well as features of the right arm. Although not depicted in further detail, merely for brevity and clarity, corresponding features of a right side, leg, midsection, and so forth may be observed and compared to determine trends indicative of an anomaly, such as a medical emergency (e.g., a stroke). The depicted features of the time-series feature set 700 include a first left wrist feature 710, first left shoulder feature 712, and first left elbow feature 714, which may be indicative of a position on the steering wheel or other vehicle control, and may further be detected by a corresponding input thereto (e.g., torque applied to the steering wheel or corresponding steering angle sensor).

Referring now to FIG. 7B, features corresponding to a second time, T₂, of the time-series feature set 700 from a second 3D point representation in the sequence from the imaging sensor are depicted. For example, the second 3D point representation may show a position of a second left wrist feature 716, second left shoulder feature 718, and second left elbow feature 720. Because movement of the left arm may appear to be functioning normally, the inactivity of the right arm could be indicative of hemiplegia or hemiparesis, such as incident to a stroke. A rate, pattern, trend, other information associated with the movement can classify the features according to one or more predefined classifications such as a magnitude of a presence of an anomaly (e.g., anomaly vs. no-anomaly classification) a magnitude of an anomaly (e.g., low, medium, or high), or a particular medical condition. Further depicted are the facial features 702 which may be employed to temporally align the body feature data with information depicted in FIG. 5 (e.g., increased detail or associated with a different sensor), or may contain further indicia of an anomaly such as an emergency medical condition. Likewise, the various body position features 704 can include information relevant to a classification of a medical event (e.g., a severity thereof). For example, sympathetic or compensatory movements of a left side may be employed to classify a stroke, or a severity thereof.

Referring now to FIG. 7C, features corresponding to a third time, T₃, of the time-series feature set 700 from a third 3D point representation in the sequence from the imaging sensor are depicted. Particularly, a third left wrist feature 722, third left shoulder feature 724, and third left elbow feature 726 are shown, along with the other body position features 704 and facial features 702 as in FIG. 7A and FIG. 7B.

Referring now to FIG. 7D, a graphical depiction of time-series data change is provided for the left wrist and elbow relative to a vertical plane. The graphical depiction is provided for ease of description. In various embodiments, any number of data structures can represent time-series data. For example, time-series rows or columns, or flattened representations thereof can be ingested by various models as described above. Such time-series data can include temporal or spatial compensation or normalization to position the data relative to a fixed location in the vehicle, another body portion or movement thereof, or a physiological parameter of a user.

A time axis 732 including the first time T₁ 736 of FIG. 7A, the second time T₂ 738 of FIG. 7B, and the third time T₃ 740 of FIG. 7C are provided along with additional times, corresponding to features which are sensed or inferred therebetween, prior to, and subsequent to such times 736, 738, 740. Particularly, a first trend line 742 depicts left shoulder position and a second trend line 744 depicts left wrist position. The positions are provided over along a time axis 732 and a relative position axis 734, provided according to an arbitrary scale indicating a vertical departure from an initial position. The position of the left elbow can be provided in another data structure or another portion of the data structure (e.g., relative to a horizontal axis, a position of a right elbow, a slackening of a jaw, a speed of eye movement, or any other data). The time-series data (e.g., according to the depicted graph, an array, etc.) can be employed to classify an anomaly, such as an existence of the anomaly, a severity of the anomaly, or a particular medical condition.

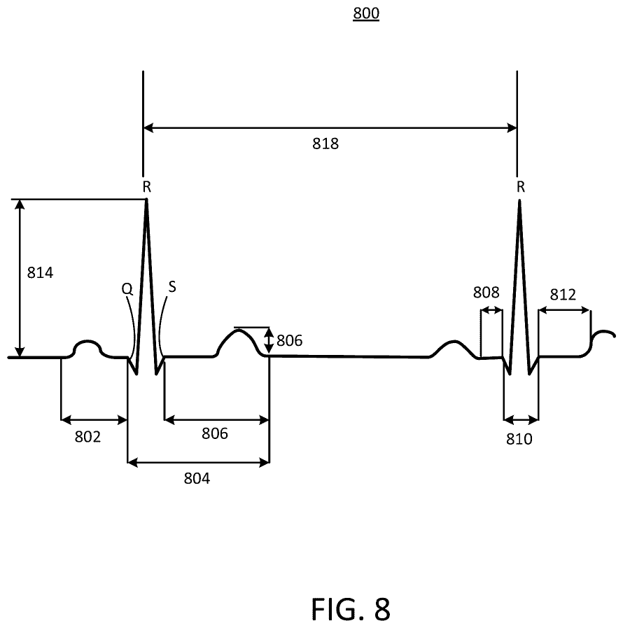

FIG. 8 depicts features associated with sensor data 122 received from a physiological sensor 106B, in accordance with some aspects. Particularly, the sensor data 122 includes an electrocardiogram 800. Features extractable from the electrocardiogram 800 can include a P-R interval 802, Q-T interval 804, S-T interval 806, P-R segment 808, QRS interval 810, and S-T segment 812, or information indicative of a heart rate 818. The systems and methods of the present disclosure can further extract an R-magnitude 814, T-wave magnitude 816, and so forth. The depicted features are intended to be illustrative and not limiting. For example, additional or fewer features may be extracted according to a predictive power (e.g., based on a correlation with an event or an availability of training data comprising the features). In various embodiments, the features may be extracted and stored for later comparison (e.g., in the case of a detected anomaly). For example, a circular buffer can store features such that upon a classification of an event such as a transition to an altered mental status, body position trend 312, or collision, the information of the circular buffer, or a subset thereof can be conveyed (e.g., by the decisioning system 114) to the server system 152 along with one or more unique or non-unique identifiers 128, and indication of the classification or other event. The server system 152 can develop iterated models based on the received data, and convey the updated models to the vehicle 102 via the network 150.

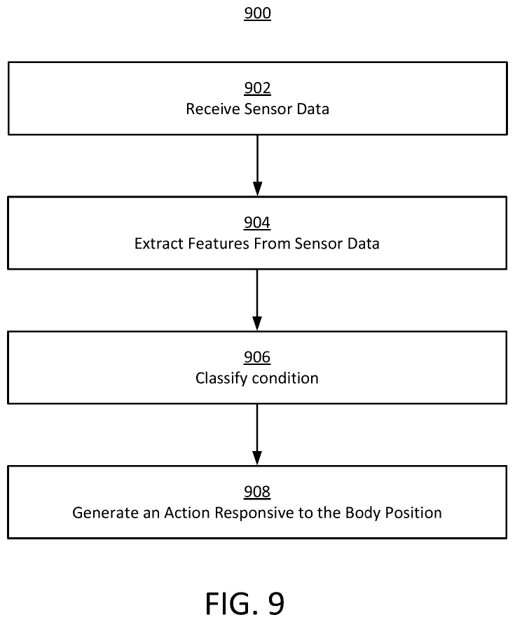

FIG. 9 depicts a flow chart for a method 900, in accordance with some aspects. The method 900 may be performed by a data processing system such as the system 100 or vehicle 102 of FIG. 1. It is noted that the method 900 is merely an example, and is not intended to limit the present disclosure. Accordingly, it is understood that additional operations may be provided before, during, and after the method 900 of FIG. 9, and that some other operations may only be briefly described herein.

In brief overview, the method 900 includes operation 902 of receiving sensor data 122. The method 900 includes operation 904, of extracting features from the sensor data 122. The method 900 includes operation 906, of classifying a body position trend 312 or associated condition 212. The method 900 includes operation 908, of generating an action responsive to the body position trend 312.

Referring again to operation 902, the method 900 includes receiving, by a data processing system, sensor data 122. The sensor data 122 can include video from an imaging sensor 106A. The video may include a data stream encoded as a same bit stream, or a time-series set of 3D point representations conveyed as separate files or bit streams (e.g., thirty frames per second, one frame per second, or so forth). The data processing system may further receive sensor data 122 such as an occupant physiological parameter. The physiological parameter can include heart rate, skin temperature, pupil dilation, or the like. The physiological parameter can be or include time-series data of a sensor 106 associated with the physiological parameter (e.g., a time-series voltage associated with an electrocardiogram from which heart rate, arrhythmia, atrial fibrillation, ventricular fibrillation, etc., can be determined). The physiological parameter can be or include processed data such as a numeric or Boolean indication of the physiological parameter. The physiological parameter can be received from a sensor 106 which is integral to the vehicle 102, or from another device interfacing therewith. For example, the physiological parameter can include data received from a mobile or wearable device.

Referring again to operation 904, the method 900 includes extracting, by a data processing system, a plurality of features of the vehicle occupant from the video. For example, the extraction of the features can include a determination of data points disposed over a facial landscape of a user. The extraction of the features can include data points disposed over a body position of a user. For example, the data processing system can generate a number of predefined data points indicative of a facial feature, body position, and so forth. Each data point may include a relative position of the vehicle occupant. For example, data points can correspond to each of an eyebrow arch, outer corner of left eye, inner corner of left eye, upper eyelid of left eye, lower eyelid of left eye, bridge of the nose, left nostril, right nostril, right shoulder height, left shoulder height, and so forth. The locations can be normalized according to a two or three-dimensional model, such as by rotating, skewing, or resizing the data points. Outliers can be removed, and deficient readings can be identified, flagged, replaced with interpolated data, or so forth.

Referring again to operation 906, the method 900 includes classifying, by a data processing system, a condition 212 based on a body position trends 312 selected from a plurality of conditions or body position trends 312. For example, the data processing system can include one or more machine learning models which can ingest the extracted features from the video at operation 904. The data processing system can receive further sensor data 122 such as a physiological parameter of an occupant of the vehicle 102. For example, the data processing system can associate physiological parameters with the features from the video (e.g., according to a temporal correlation), and ingest features extracted from the associated physiological parameters in a same data structure as the features of the video data. The machine learning models can classify a condition or associated body position trend 312, based on the features extracted from the video and from other sensors 106 (or classify the information as corresponding with a lack of an anomaly, an insufficiency of information, or so forth).

Referring again to operation 908, the method 900 includes generating, by a data processing system, an action in response to the classification. For example, the action can be a navigational action such as bringing the vehicle 102 to a halt (e.g., immediately applying the brakes, or applying the brakes in combination with a steering system to remove a vehicle 102 from an active portion of a roadway, or so forth), or causing the vehicle 102 to travel to a medical provider. The action can be a message to a decisioning system 114 to cause the decisioning system 114 to effectuate the action. The action can be a non-navigational action such as providing an indication to an occupant or non-occupant, such as to alert medical personal or a caretaker of a medical event, to alert the user of their condition 212, or so forth. The action can be determined by the decisioning system 114, and thereafter, conveyed by communications circuit 114A.

FIG. 10 depicts another flow chart for a method, in accordance with some aspects. The method 1000 may be performed by data processing systems such as the system 100 or vehicle 102 of FIG. 1. It is noted that the method 1000 is merely an example, and is not intended to limit the present disclosure. For example, a corresponding method can be employed to determine whether another condition detected based on a body position trend 312 is maskable. Accordingly, it is understood that additional operations may be provided before, during, and after the method 1000 of FIG. 10, and that some other operations may only be briefly described herein. References to confidences or confidence levels can refer to a confidence along a spectrum, or a Boolean indication of user acknowledgement or non-acknowledgement.

In brief overview, the method 1000 includes operation 1002 of receiving a classification. The method 1000 includes operation 1004, of determining if the classification is maskable. The method 1000 includes operation 1006, of providing a prompt to a user. Based on a response received at operation 1008, the method 1000 can proceed to operation 1010, to generate an action, or await a further classification at operation 1002.

Referring again to operation 1002, the method 1000 includes receiving a classification, such as a classification for an altered mental status. For example, the altered mental status can be received incident to the classification of the altered mental status as described above, with regard to operation 906.

Referring again to operation 1004, the method 1000 includes determining if the classification is maskable. The maskability can depend on a predetermined condition or input to a user interface 108. For example, a user can indicate, prior to a system 100 performing the method 1000, that one or more conditions or altered mental statuses should be maskable (or disabled) such as in response to a precious false positive. In some embodiments, a user interface 108 does not provide a selection of maskability or disabling a function. In some embodiments, the user interface 108 includes a display only feature to prevent interaction between the systems and methods of the present disclosure and the decisioning system 114. The maskability determination can depend on a confidence level 314, severity, or type of condition 212 or altered mental status. For example, a condition 212 such as hypoglycemia can lead to confusion such that a non-maskability may be selected (e.g., because an occupant may not believe themselves to be experiencing an altered mental status). A mental status such as unconsciousness incident to heart attack may be maskable, since an occupant indicating a request to mask such a condition 212 is unlikely to be experiencing the condition 212. Further, maskability or an action taken may depend on a number of occupants in a vehicle 102 (multiple users may be alerted to a condition 212, a likelihood of all users being confused about the condition 212 may be reduced).

Referring again to operation 1006, the method 1000 includes providing a prompt to a user. The prompt can be provided via an audible notification, a touchscreen display, or so forth. The user can be a vehicle occupant or non-occupant. The prompt may provide an indication to take an action, provide a response, or may be informational.

An illustrative example of a prompt of operation 1006 is provided by reference to the prompt 1102 of FIG. 10, and may be provided, for example, via a user interface 108 including a GUI of a vehicle 102, such as a GUI of an infotainment display, a dashboard, or a tablet, mobile phone, or other device in network communication with the vehicle 102. As depicted, the user interface 108 can provide the prompt 1102 along with one or more selections for user response, such an explicit affirmative response 1104, an explicit negative response 1106, shown provided along with an indication of an action associated with the response, and another response 1108, such as a response 1108 associated with a timeout.

Referring again to operation 1008, the method 1000 includes receiving a response to the prompt of operation 1006. The response can include a spoken response, selection of a button or touchscreen, movement of a face into a field of view of an imaging sensor 106A, or so forth. For example, the response can include an indication of the explicit affirmative response 1104 or explicit negative response 1106 of FIG. 10. The response can be an expiration of a timeout without a user providing an affirmative indication, such as the response 1108 shown associated with the timeout shown in FIG. 10. The response can include one or more discrete responses, such as an indication to or not to engage a decisioning system 114, or a response indicating a presence or absence of a condition 212 associated with a potential altered mental status. The response can include associated information such as a delay between the prompt and the response, parametric information of a spoken response indicative of a condition 212, or so forth. For example, a user selection of a non-active portion 1110 of a graphical user interface may be indicative of confusion or loss of motor control of a vehicle occupant. In some embodiments, the method can proceed to operation 1010 based on such information. Responsive to a lack of a receipt of an explicit response (e.g., upon a time-out expiration), the method 1000 can proceed to operation 1010. Responsive to an explicit response, the method 1000 can await a further classification of an altered mental status, at operation 1002.

Referring again to operation 1010, the method 1000 includes generating an action. For example, the action can include a navigational action or non-navigational action such an audible or visual alert, or a conveyance of a message, as is further described with reference to operation 908 of FIG. 9.

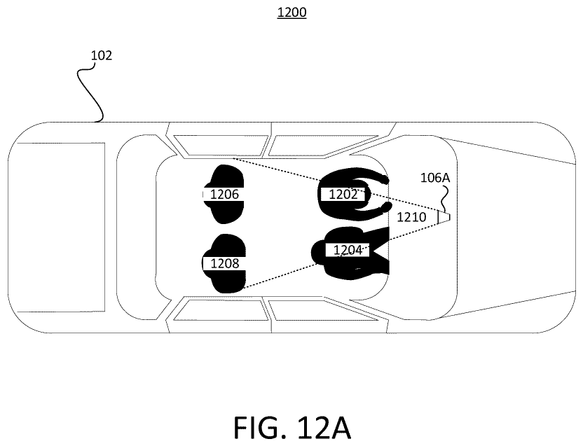

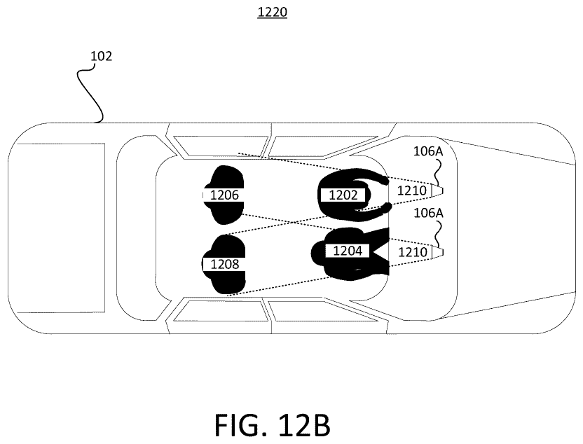

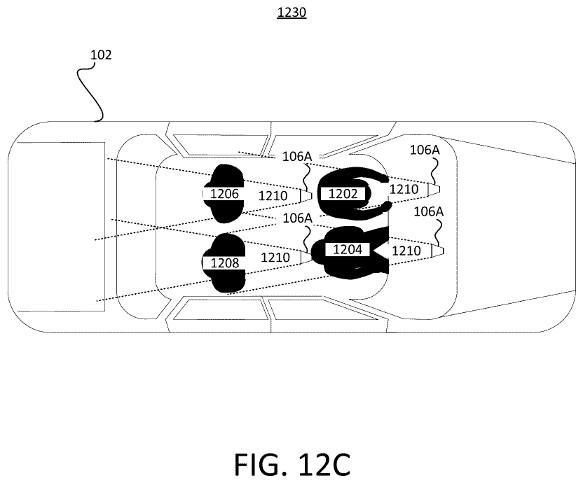

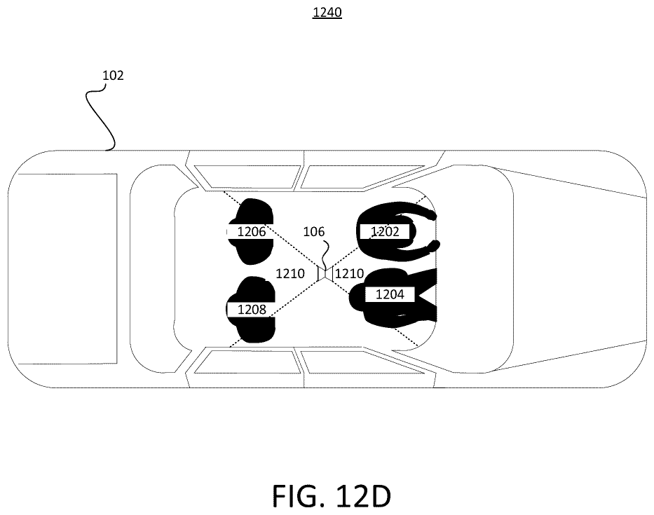

FIG. 12A is a top view 1200 of a vehicle 102 including various occupants. For example, a first occupant 1202 can be a vehicle operator, and a second occupant 1204 can be a vehicle passenger, along with a third occupant 1206 and fourth occupant 1208. The various systems and methods herein can be employed with regard to the various occupants of the vehicle, which may aid medical care provisioned to one or more vehicle occupants 1202, 1204, 1206, 1208. As depicted, a sensor 106 (e.g., the depicted imaging sensor 106A) can be disposed on a dashboard, rear view mirror, or other position of the vehicle, having a field of view (FOV) 1210 including the vehicle occupants 1202, 1204, 1206, 1208. The sensor 106 can reduce device complexity or cost, establish a line of sight with various vehicle occupants, and may be co-located with existing sensors (e.g., a front facing camera of a vehicle autonomy system 114B).

Referring now to FIG. 12B, another top view 1220 of a vehicle 102 including the occupants of FIG. 12A is provided. As depicted, a sensor 106 (e.g., the depicted imaging sensors 106A) is positioned in front of each of the front occupants of the vehicle 102. Such a sensor 106 may include a lesser FOV 1210, relative to the FOV 1210 of the sensor 106 of FIG. 12A, and may reduce disaggregation between, for example, the first occupant 1202 and a second occupant 1204 of the vehicle 102. In various embodiments, the imaging sensors 106A can monitor either of the front seat occupants 1202, 1204 or the back seat occupants 1206, 1208. Referring now to FIG. 12C, another top view 1230 of a vehicle 102 including the occupants of FIG. 12A is provided. As depicted, sensors 106 (e.g., the depicted imaging sensors 106A) are placed in front of each occupant. Such sensors 106 can include a separate data stream for each occupant 1202, 1204, 1206, 1208 and may capture further vehicle information or context.

Referring now to FIG. 12D, another top view 1240 of a vehicle 102 including the occupants of FIG. 12A is provided. As depicted, further sensors 106 may include non-imaging sensors 106 such as physiological sensors 106B for the occupants. As depicted, sensors 106 can be collocated or include separate FOVs 1210. For example, the sensors 106 of FIG. 12D can include a line of sight to a body (and/or face) of each occupant 1202, 1204, 1206, 1208 (e.g., from a vehicle ceiling, seatback, etc.). In some configurations, the sensors 106 may include a line of sight to a face, chest, upper body, and/or other body portions, and one or more sensors can be configured for particular body portions such that an occupant may be observed by one or more sensors 106. Such positioning or co-location may further ease installation and increase spatial diversity between the occupants 1202, 1204, 1206, 1208 to ease disaggregation of sensor data 122 corresponding thereto. For example, sensors 106 such as millimeter wave radar detectors, thermal cameras, or the like can observe occupants. In some embodiments, the four separate sensor FOV 1210 can be located at the depicted position (e.g., four sensors 106 of an assembly). In some embodiments, a front facing FOV 1210 and rear facing FOV 1210 can be intermediate to each of the first occupant 1202 and third occupant 1206, or second occupant 1204 and fourth occupant 1208. Such sensors 106 may further disaggregate occupants, by including a separate sensor 106 or FOV 1210 for each.

FIG. 13 is a block diagram illustrating an architecture for a computer system 1300 that can be employed to implement elements of the systems and methods described and illustrated herein. The computer system or computing device 1300 can include or be used to implement a data processing system or its components, and components thereof. The computing system 1300 includes at least one bus 1305 or other communication component for communicating information and at least one processor 1310 or processing circuit coupled to the bus 1305 for processing information. The computing system 1300 can also include one or more processors 1310 or processing circuits coupled to the bus for processing information. The computing system 1300 also includes at least one main memory 1315, such as a random-access memory (RAM) or other dynamic storage device, coupled to the bus 1305 for storing information, and instructions to be executed by the processor 1310. The main memory 1315 can be used for storing information during execution of instructions by the processor 1310. The computing system 1300 may further include at least one read only memory (ROM) 1320 or other static storage device coupled to the bus 1305 for storing static information and instructions for the processor 1310. A storage device 1325, such as a solid status device, magnetic disk or optical disk, can be coupled to the bus 1305 to persistently store information and instructions (e.g., for the data repository 120).

The computing system 1300 may be coupled via the bus 1305 to a display 1335, such as a liquid crystal display, or active-matrix display. An input device 1330, such as a keyboard or mouse may be coupled to the bus 1305 for communicating information and commands to the processor 1310. The input device 1330 can include a touch screen display 1335.

The processes, systems and methods described herein can be implemented by the computing system 1300 in response to the processor 1310 executing an arrangement of instructions contained in main memory 1315. Such instructions can be read into main memory 1315 from another computer-readable medium, such as the storage device 1325. Execution of the arrangement of instructions contained in main memory 1315 causes the computing system 1300 to perform the illustrative processes described herein. One or more processors in a multi-processing arrangement may also be employed to execute the instructions contained in main memory 1315. Hard-wired circuitry can be used in place of or in combination with software instructions together with the systems and methods described herein. Systems and methods described herein are not limited to any specific combination of hardware circuitry and software.

Although an example computing system has been described in FIG. 13, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer-based components.

The systems described above can provide multiple ones of any or each of those components and these components can be provided on either a standalone system or on multiple instantiations in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C#, PROLOG, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient status configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses (or systems). Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices include cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network 150.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface 108 or a web browser through which a subject can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A system, comprising:
a data processing system comprising one or more processors coupled with memory, the data processing system configured to:
receive sensor data from a plurality of sensors associated with a vehicle, the plurality of sensors comprising an imaging sensor and the sensor data comprising images or 3D point representations of a vehicle occupant;
extract a plurality of time-series features of the vehicle occupant from the images or 3D point representations;
execute a machine learning model using the plurality of time-series features of the vehicle occupant to determine at least one condition of the vehicle occupant inferred from the plurality of time-series features; and
responsive to the condition, generate an instruction to cause the vehicle to perform a navigational action.

2. The system of claim 1, wherein the instruction is configured to:
convey a message comprising the condition to a user interface.

3. The system of claim 2, wherein the message comprises an indication of the determined condition of an occupant of the vehicle.

4. The system of claim 2, wherein the navigational action comprises bringing the vehicle to a halt.

5. The system of claim 1, wherein the data processing system is further configured to:
convey, via a user interface, a prompt for input,
wherein the performance of the navigational action is based on a response to the prompt.

6. The system of claim 5, wherein the prompt is conveyed to the vehicle occupant.

7. The system of claim 6, wherein the machine learning model is further trained to determine a different condition, and the data processing system is further configured to:
generate the instruction to cause the vehicle to perform the navigational action without conveying the prompt to the vehicle occupant.

8. The system of claim 5, wherein the prompt is conveyed to a non-occupant of the vehicle.

9. The system of claim 1, wherein the data processing system is further configured to:
temporally align the sensor data for ingestion by the machine learning model;
determine that the sensor data includes deficient sensor data; and
generate replacement sensor data for the deficient sensor data, prior to ingestion by the machine learning model.

10. The system of claim 1, wherein the sensor data includes:
a physiological parameter for the vehicle occupant;
a user parameter, a vehicle parameter, and an association therebetween, and
wherein the determination of the condition is based on the physiological parameter and the association between the user parameter and the vehicle parameter.

11. The system of claim 1, wherein the classification is based on a prior medical history of the vehicle occupant.

12. A vehicle, comprising one or more processors coupled with memory, the one or more processors configured to:
receive sensor data from a plurality of sensors associated with the vehicle, the plurality of sensors comprising an imaging sensor and the sensor data comprising images or 3D point representations of a vehicle occupant;
extract a plurality of time-series features of the vehicle occupant from the images or 3D point representations;
execute a machine learning model using the plurality of time-series features of the vehicle occupant to determine at least one condition of the vehicle occupant inferred from the plurality of time-series features; and
responsive to the condition, convey a message indicative of the condition for presentation by a user interface.

13. The vehicle of claim 12, wherein the vehicle is further configured to:
generate an instruction to cause the vehicle to perform a navigational action based on the condition.

14. The vehicle of claim 12, wherein the vehicle is configured to:
convey, via the user interface, a prompt for input,
wherein the message is conveyed based on a response to the prompt.

15. The vehicle of claim 14, wherein the vehicle is further trained to:
determine a different condition; and
convey the message without conveying the prompt for input.

16. The vehicle of claim 12, wherein the vehicle is configured to:
temporally align the sensor data for ingestion by the machine learning model;
determine that the sensor data includes deficient sensor data; and
generate replacement sensor data for the deficient sensor data, prior to ingestion by the machine learning model.

17. The vehicle of claim 12, wherein the sensor data comprises a user parameter, a vehicle parameter, and an association therebetween.

18. A method comprising:
receiving, by a data processing system, sensor data from a plurality of sensors associated with a vehicle, the plurality of sensors comprising an imaging sensor and the sensor data comprising images or 3D point representations of a vehicle occupant;
extracting, a plurality of time-series features of the vehicle occupant from the images or 3D point representations;
executing, by the data processing system, a machine learning model using the plurality of time-series features of the vehicle occupant to determine at least one condition of the vehicle occupant inferred from the plurality of time-series features; and
generating, by the data processing system, an instruction to cause the vehicle to perform a navigational action, responsive to the determination of the condition.

19. The method of claim 18, further comprising:
conveying, by the data processing system, a prompt for input,
wherein the performance of the navigational action is based on a response to the prompt.

20. The method of claim 18, further comprising:
presenting, by the data processing system, an indication of the navigational action and the condition to an occupant of the vehicle; and
conveying, by the data processing system, a message indicative of the condition to a non-occupant of the vehicle.
